(19) United States
(12) Patent Application Publication
(10) Pub. No.: US 2010/0057171 A1
(43) Pub. Date: Mar. 4, 2010

(54) MOBILE APPARATUS FOR PROVIDING CRYOTHERAPY AND THERMOTHERAPY TO A REGION OF A KNEE BEING IN ELEVATED POSITION

(76) Inventor: TRISHA STEPHENS, Orlando, FL (US)

Correspondence Address: James Ray & Associates, 2640 Pitcairn Road, Monroeville, PA, 15146, US

(21) Appl. No.: 12/550,641

(22) Filed: Aug. 31, 2009

Related U.S. Application Data
(60) Provisional application No. 61/093,813, filed on Sep. 3, 2008.

(51) Int. Cl. A61F 7/00; A61F 7/08; A61F 7/10

(52) U.S. Cl. 607/108; 607/112

(57) ABSTRACT

An apparatus for applying at least one of cryotherapy and thermotherapy to at least a portion of a knee region of an elevated leg includes a tripod and a knee support member attached to a top end of the tripod and having a predetermined length and a generally U-shaped cross-section in a direction transverse to the predetermined length, the U-shaped cross-section defined by each of an arcuate portion mounted on a top end of the first member and a pair of spaced apart legs disposed in a generally vertical plane and upstanding on the arcuate portion. A pouch is engageable with each of the knee support member and the at least portion of the knee region and is adapted for holding an ice pack or a heat pack therewithin for applying at least one of cold and heat to the knee region.

### CROSS-REFERENCE TO RELATED APPLICATIONS

This application is related to and claims priority from Provisional Patent Application Ser. No. 61/093,813 filed on Sep. 3, 2008.

### FIELD OF THE INVENTION

The present invention relates, in general, to cryotherapy and/or thermotherapy and, more particularly, this invention relates to an apparatus for applying at least one of cryotherapy and thermotherapy to at least a portion of a knee region of a leg being in elevated position.

### STATEMENT REGARDING FEDERALLY SPONSORED RESEARCH AND DEVELOPMENT

N/A

### REFERENCE TO SEQUENCE LISTING, A TABLE, OR A COMPUTER PROGRAM LISTING COMPACT DISC APPENDIX

N/A

### BACKGROUND OF THE INVENTION

As is generally well known, knee injuries may take an extended period of time to heal depending on the severity of the injury and nature of the rehabilitation process. It is also generally well known that the knee should be kept in an elevated position to facilitate healing process. Furthermore, application of ice and/or heat is effective therapy, particularly when the injury occurs or aggravated. However, many individuals who damage their joints, ligaments and other parts of their knee as well as muscles in regions of the leg adjoining the knee, cannot afford to take time off work or slow down their lives in order to keep the knee in the elevated position.

Prior to the conception and design of the present invention various devices have been in use for supporting the knee in the elevated position, however none of the devices, to the best knowledge of the inventor, are provided for applying cryotherapy or thermotherapy to the knee region.

### SUMMARY OF THE INVENTION

The invention provides an apparatus for applying at least one of cryotherapy and thermotherapy to at least a portion of a knee region of an elevated leg. The apparatus includes a tripod and a knee support member attached to a top end of the tripod. The knee support member has a predetermined length and a generally U-shaped cross-section in a direction transverse to the predetermined length. The U-shaped cross-section is defined by each of an arcuate portion mounted on a top end of the first member and a pair of spaced apart legs disposed in a generally vertical plane and upstanding on the arcuate portion. A contoured inner surface may be carried on the arcuate portion. The contoured inner surface has a generally convex shape in each of the generally vertical plane and in a direction parallel to the predetermined length. At least a portion of the arcuate portion carrying the contoured inner surface thereon is manufactured from a combination of cork and latex materials enhanced with a layer of memory foam. A pouch is engageable with each of the knee support member and the at least portion of the knee region. The pouch has each of a hollow interior and an opening formed through a wall or edge of the pouch for inserting into the hollow interior or removing therefrom at least one of a cold pack, a heat pack and a gel pack. A fastener is provided for selectively opening and closing the opening in the pouch. There is also a hinge securing one end of the pouch to a free end of one of the pair of legs.

### OBJECTS OF THE INVENTION

It is, therefore, one of the primary objects of the present invention to provide an apparatus for applying at least one of cryotherapy and thermotherapy to at least a portion of a knee region being in elevated position.

Another object of the present invention is to provide an apparatus for applying at least one of cryotherapy and thermotherapy to at least a portion of a knee region of a leg being in elevated position that has tripod base with adjustable legs.

Yet another object of the present invention is to provide an apparatus for applying at least one of cryotherapy and thermotherapy to at least a portion of a knee region of a leg being in elevated position that includes at least one pouch capable of holding icepack or heat pack.

A further object of the present invention is to provide an apparatus for applying at least one of cryotherapy and thermotherapy to at least a portion of a knee region of a leg being in elevated position that includes a detachable gel pack.

Yet a further object of the present invention is to provide an apparatus for applying at least one of cryotherapy and thermotherapy to at least a portion of a knee region of a leg being in elevated position that includes a gel pack and a Peltier device connected to the gel pack.

An additional object of the present invention is to provide an apparatus for applying at least one of cryotherapy and thermotherapy to at least a portion of a knee region of a leg being in elevated position that includes a convex portion for facilitating support of the knee.

Another object of the present invention is to provide an apparatus for applying at least one of cryotherapy and thermotherapy to at least a portion of a knee region of a leg being in elevated position that is simple to use.

A further object of the present invention is to provide an apparatus for applying at least one of cryotherapy and thermotherapy to at least a portion of a knee region of a leg being in elevated position that is economical to manufacture.

In addition to the several objects and advantages of the present invention which have been described with some degree of specificity above, various other objects and advantages of the invention will become more readily apparent to those persons who are skilled in the relevant art, particularly, when such description is taken in conjunction with the attached drawing Figures and with the appended claims.

### BRIEF DESCRIPTION OF THE DRAWINGS

FIG. 1 is an isometric view of an apparatus for applying at least one of cryotherapy and thermotherapy to at least a portion of a knee region being in elevated position;

FIG. 2 is a partial end view of the apparatus of FIG. 1;

FIG. 3 is a partial isometric view of the apparatus of FIG. 1, particularly illustrating a pouch for holding an ice or heart pack;

FIG. 4 is one environmental view of using the apparatus of FIG. 1;

FIG. 5 is another environmental view of using the apparatus of FIG. 1;

FIG. 6 is a partial end view of the apparatus of FIG. 1 particularly illustrating a pair of pouches each for holding an ice or heart pack;

FIG. 7 is a partial end view of the apparatus of FIG. 1 particularly illustrating an integral gel pack;

FIG. 8 is a partial end view of the apparatus of FIG. 1 particularly illustrating a detachable gel pack; and

FIG. 9 is a partial end view of the apparatus of FIG. 1 particularly illustrating a gel pack with a Peltier device.

### BRIEF DESCRIPTION OF THE VARIOUS EMBODIMENTS OF THE INVENTION

Prior to proceeding to the more detailed description of the present invention, it should be noted that, for the sake of clarity and understanding, identical components which have identical functions have been identified with identical reference numerals throughout the several views illustrated in the drawing figures.

It is to be understood that the definition of a knee applies to the joint between the thigh and the lower leg, formed by the articulation of the femur and the tibia and covered anteriorly by the patella and that the definition of a knee region applies to such knee joint with the adjoining portions of the upper and lower leg.

The present invention describes an apparatus, generally designated as 10, which supports a knee region 4 of a leg 2 being disposed in elevated position and which provides means to apply at least one of cryotherapy and thermotherapy to at least a portion of the knee region 2.

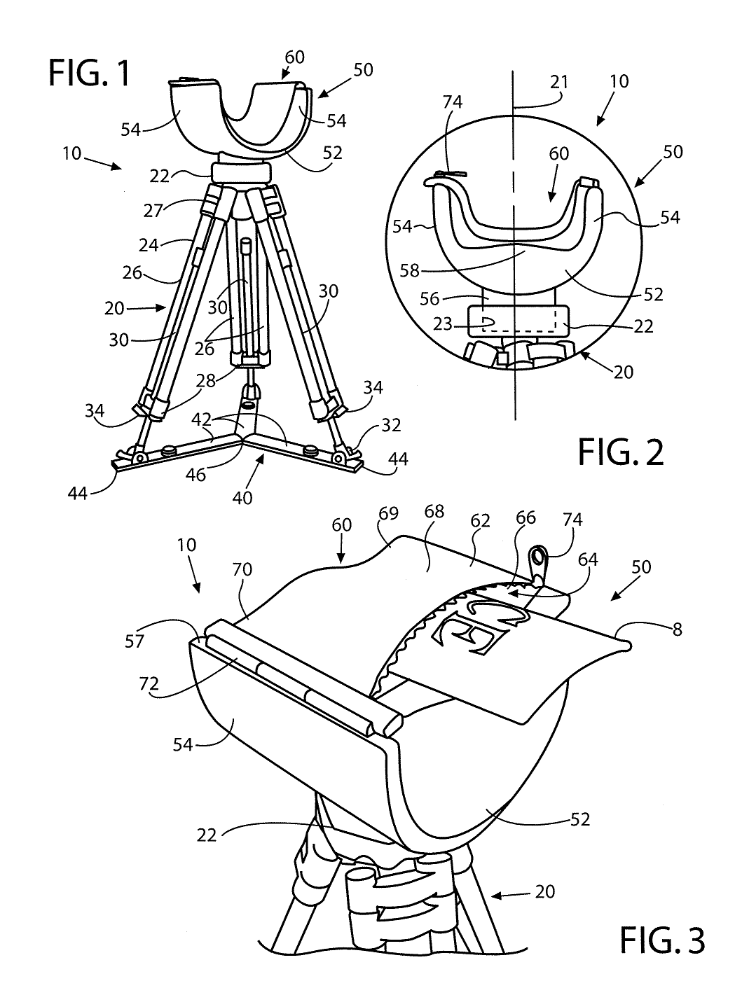

Reference is now made to FIG. 1, wherein the apparatus 10 is shown as including a first member, generally designated as 20. Preferably, such first member 20 is a tripod having a top end 22 and legs 24 extending downwardly therefrom. It is further preferred that each leg 24 is of an adjustably telescopic type. Although each leg 24 has been illustrated in FIG. 1 as having a pair of spaced apart stationary members 26 joined together with a pair of end fittings 27, 28 and a movable member 30 disposed between stationary members 26 for telescopic movement relative thereto, the leg 24 may be of any conventional telescopic type. A distal end of the movable member 30 carries thereon a bottom end 32 of the leg 24. A triad of locking devices 34 is also provided for fixing the top end 22 of the tripod 20 at a preselected height. By way of an example only of FIG. 1, each locking device 34 is provided in combination with a lower end fitting 28 of each leg 24. Use of the tripod 20 enables ease of handling and mobility of the apparatus 10.

An optional spreader 40 is also contemplated for controlling spaced position of the bottom end 32 of each leg 24 relative to vertical axis of the tripod 20. By way of an example only, the spreader 40 is shown in FIG. 1 as having a triad of elongated members 42 joined at one end thereof, wherein a distal end 44 of each elongated member 42 is detachably and lockably attached to the bottom end 32 of a respective leg 24 and wherein the spreader 40 is foldable about its center joint 46. Such spreader 40 may be of a type as manufactured by Lino Manfrotto & Co., S.p.a. of Italy under model number 3138.

Now, more particularly, to FIGS. 1-3, the apparatus 10 also includes a second member, generally designated as 50. Such second member 50 is essentially a cradle for supporting the knee and has a predetermined length and a generally U-shaped cross-section in a direction transverse to the predetermined length. The U-shaped cross-section is defined by each of an arcuate portion 52 mounted on the top end 22 of the tripod 20 and a pair of spaced apart legs 54 upstanding on the arcuate portion 52 and disposed, generally parallel to one another, in a generally vertical plane. The second member 50 may be attached to the top end 22 of the tripod 20 in a stationary manner, however, it is presently preferred to allow the second member 50 to rotate in a horizontal plane about the vertical axis 21. Such rotation may be achieved, by way of an example of FIG. 2, wherein the end 22 is adapted with a round bore 23 and wherein a shaft portion 56 is provided on the outer surface of the arcuate portion 52. The round cross-sectional shape of the shaft portion 56 is sized to be received within the bore 23 so as to permit rotation of the shaft portion 56 within the bore 23 about the axis 21.

The second member 50 may be manufactured with varying length and the cross-sectional shape in order to facilitate support of a knee of a particular user.

The final essential element of the apparatus 10 is means, generally designated as 60, engageable with each of the second member 50 and the at least portion of the knee region 4 for applying at least one of cold and heat thereto.

Such means 60 may include a pouch 62 having a hollow interior 64 and an opening (or slit) 66 formed through a wall 68 or edge portion 69 of the pouch 62 for inserting into the hollow interior 64 or removing therefrom at least one of a cold pack and a heat pack 8. One end 70 of the pouch 62 is secured to a free end 57 of one of the pair of legs 54. Preferably, a hinge member 72 is provided for securing such one end 70. The present invention also contemplates a closure means for selectively opening and closing the opening 66. Such closure means is illustrated in FIG. 3 as a zipper fastener 74 but may be of any other suitable type, for example such as a hook and loop fastener (not shown), for closing an elongated opening 66. The pouch 62 is made from any suitable material allowing transfer of cold or heat from ice or heat pack 8 respectively for application onto the knee region 4.

Preferably, the length of the pouch 62 in a direction transverse to the predetermined length of the second member 50 is set so as to allow application of cryotherapy or thermotherapy to the side portions of the knee region 4. The width of the pouch 62 in the direction parallel to the predetermined length of the second member 50 is generally set to at least cover the knee joint and is preferably set to cover the knee region 4.

In combination with the pouch 62, the arcuate portion 52 may be provided with a contoured inner surface 58 having a generally convex shape in each of the vertical plane and in a direction parallel to the predetermined length of the second member 50. A convex shape of such contoured inner surface 58 is provided so as to closely match the contour of the rear portion of the knee region 4. Such contoured inner surface 58 has been found advantageous in improving ergonomics of supporting the knee region 4 and, more importantly, increasing the surface contact area. It has been further found advantageous to manufacture at least a portion of such arcuate portion 52 carrying the contoured inner surface 58 thereon from a combination of cork and latex materials enhanced with a layer of memory foam. Such material is available under the Birko-Flor® brand. Such material molds and shapes over time to the rear and side portions of the knee 4 due to the body heat, thus creating a custom fit depending on the shape and size of the knee 4.

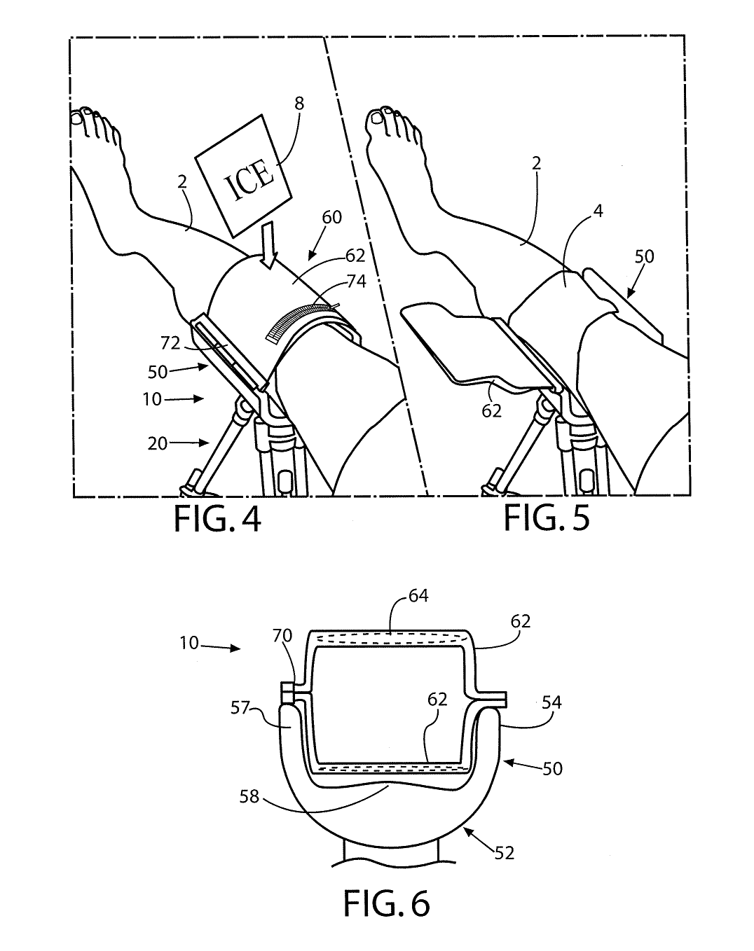

The use of the apparatus 10 of FIGS. 1-3 is illustrated in FIGS. 4-5 for applying cryotherapy to the top or front portion of the knee region 4. FIG. 4 shows a pouch 62 with ice pack 8 being operatively positioned over the knee region 4, while FIG. 5 shows the pouch 62 being rotated outwardly so as to allow insertion of the knee region 4 into the second member 50 or removal of the knee region 4 therefrom. It would be appreciated that the pouch 62 as shown in FIGS. 1-2 is suitable for applying one of cryotherapy and thermotherapy to at least a rear portion of the knee region 4.

In order to treat at least the combination of the front and rear portions of the knee region 4, it is contemplated to provide a pair of pouches 62, as shown in FIG. 6, wherein each of the pair of pouches 62 has each of one end 70 thereof secured to a free end 57 of one of said pair of legs 54, a hollow interior 64 and an opening 66 formed through a wall 68 or edge portion 69 of the pouch for inserting into the hollow interior 64 or removing therefrom at least one of a cold pack and a heat pack 8. Thus, the user simply positions the knee region 4 between the pair of pouches 62.

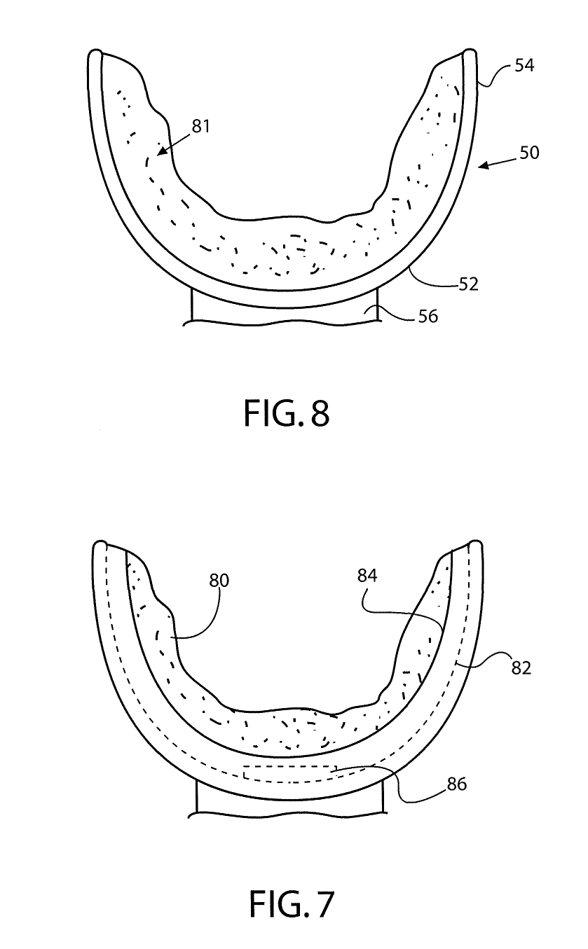

The means 60 may include a gel pack 80 at least abuttingly engageable with an interior surface of the second member 50. The gel pack 80 may be selected from a family of gel packs having a flexible shape and containing a filler material mixed with a high thermal conductivity heat transfer material. The gel pack 80 may be simply placed into the second member 50 so that one surface 82 of the gel pack 80 abuttingly engages inner surface of the second member 50, as best shown in FIG. 7. Optional retaining means, for example such as an upstanding flange 84 may be provided at each end of the second member 50 so as to prevent shifting of the gel pack 80 beyond the boundary of the second member 50. Such retaining means may also include at least one hook and loop type fastener 86 either in combination or independently from the upstanding flanges 84. It is also contemplated that the gel pack 81 may be provided integral with the inner surface of the second member 50, as best shown in FIG. 8, so that the gel pack 81 and the second member 50 are detached from the tripod 20 as a single unit and are either cooled or heated for applying cryotherapy or thermo therapy respectively.

The present invention further contemplates that a thermoelectric assembly 90 having at least one Peltier device 92 is disposed within the second member 50 in operative engagement with one surface 82 of the gel pack 80. For example, the second member 50 may be provided with an internal cavity 59 axially disposed at least within the arcuate portion 52 and may be further disposed within the shaft portion 56.

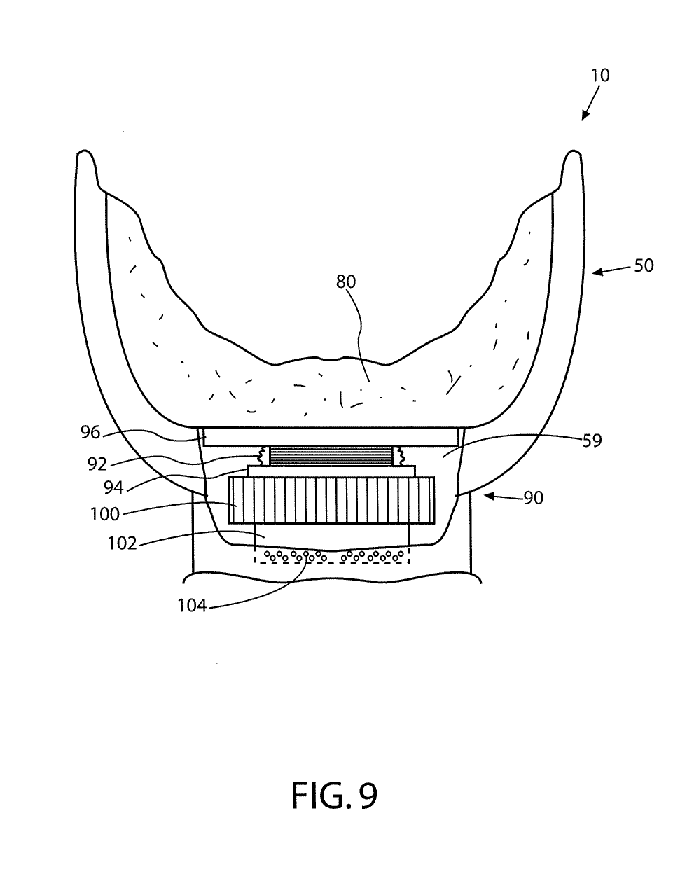

Such thermoelectric assembly 90 may be constructed in accordance with disclosure in U.S. Pat. No. 5,800,490, whose teaching are incorporated into this document by reference thereto. Briefly, as best shown in FIG. 9, in addition to the at least one Peltier device 92, such thermoelectric assembly 90 includes, a first plate 94 attached to a Peltier warm surface and a second plate 96 attached to a Peltier cool surface, whereby the gel pack 80 is abuttingly engageable with the second plate 96, a radiator 100 affixed to the first plate 94 on a first surface thereof, the radiator 100 having a fan 102 in close proximity for applying air flow and encouraging the transfer of heat energy from the radiator 100 to ambient air through orifices or apertures 104 formed through the second member 50 and a power and circuit (not shown) for controlling the Peltier device 92 and applying power to the thermoelectric assembly 90.

Although the present invention has been shown in terms of the first member 20 being a tripod, it will be apparent to those skilled in the art, that the present invention may be used with the first member being of single leg type, for example as shown in U.S. Pat. No. 2,473,94, whose teaching are incorporated into this document by reference thereto.

Furthermore, the gel pack 80 may be used as the cold or heat pack 8 and the second pouch 62 may be attached to the opposed leg 54 with a second hinge 72.

Thus, the present invention has been described in such full, clear, concise and exact terms as to enable any person skilled in the art to which it pertains to make and use the same. It will be understood that variations, modifications, equivalents and substitutions for components of the specifically described embodiments of the invention may be made by those skilled in the art without departing from the spirit and scope of the invention as set forth in the appended claims.

I claim:

1. An apparatus for applying at least one of cryotherapy and thermotherapy to at least a portion of a knee region of an elevated leg, said apparatus comprising:
(a) a first member;
(b) a second member having a predetermined length and a generally U-shaped cross-section in a direction transverse to said predetermined length, said U-shaped cross-section defined by each of an arcuate portion mounted on a top end of said first member and a pair of spaced apart legs disposed in a generally vertical plane and upstanding on said arcuate portion; and
(c) means engageable with each of said second member and the at least portion of the knee region for applying at least one of cold and heat thereto.

2. The apparatus, according to claim 1, wherein said first member is a tripod.

3. The apparatus, according to claim 2, wherein said tripod has adjustable legs.

4. The apparatus, according to claim 2, wherein said tripod includes a spreader member attached to a free end of each leg.

5. The apparatus, according to claim 1, wherein said arcuate portion of said second member includes a contoured inner surface having a generally convex shape in each of said vertical plane and in a direction parallel to said predetermined length.

6. The apparatus, according to claim 1, wherein said second member has at least an inner portion thereof manufactured from a combination of cork and latex materials enhanced with a layer of memory foam.

7. The apparatus, according to claim 1, wherein said means for applying said at least one of cold and heat includes a pouch having each of one end thereof secured to a free end of one of said pair of legs, a hollow interior and an opening formed through a wall or edge of said pouch for inserting into said hollow interior or removing therefrom at least one of a cold pack and a heat pack.

8. The apparatus, according to claim 7, wherein said means for applying said at least one of cold and heat further includes a closure means for selectively opening and closing said opening.

9. The apparatus, according to claim 8, wherein said closure means is a fastener.

10. The apparatus, according to claim 1, wherein said means for applying said at least one of cold and heat includes a pair of pouches, each of said pair of pouches having each of one end thereof secured to a free end of one of said pair of legs, a hollow interior and an opening formed through a wall or edge of said pouch for inserting into said hollow interior or removing therefrom at least one of a cold pack and a heat pack.

11. The apparatus, according to claim 1, wherein said means for applying said at least one of cold and heat includes a gel pack at least abuttingly engageable with an interior surface of said second member.

12. The apparatus, according to claim 11, wherein said gel pack is selected from a family of gel packs having a flexible shape and containing a filler material mixed with a high thermal conductivity heat transfer material.

13. The apparatus, according to claim 11, wherein said means for applying said at least one of cold and heat further includes means for retaining said gel pack within said second member.

14. The apparatus, according to claim 11, wherein said means for applying said at least one of cold and heat includes a gel pack at least one of securely attached to and formed integral with an interior surface of said second member.

15. The apparatus, according to claim 11, wherein said means for applying said at least one of cold and heat further includes a thermoelectric assembly disposed within said second member and having at least one Peltier device, a first plate attached to a Peltier warm surface and a second plate attached to a Peltier cool surface, whereby said gel pack is attached to said second plate, a radiator affixed to said first plate on a first surface, said radiator having a fan in close proximity for applying air flow and encouraging the transfer of heat energy from said radiator to ambient air, a control circuit for controlling said Peltier device, feedback means for reporting feedback signals to said control circuit for adjusting said Peltier device and a power supply for applying electrical power to said thermoelectric assembly.

16. A mobile apparatus for applying at least one of cryotherapy and thermotherapy to at least a portion of a knee region of an elevated leg, said apparatus comprising:
(a) a tripod having adjustable legs;
(b) a spreader controlling spaced position of said adjustable legs;
(c) a knee support member having a predetermined length and a generally U-shaped cross-section in a direction transverse to said predetermined length, said U-shaped cross-section defined by each of an arcuate portion mounted on a top end of said tripod and a pair of spaced apart legs upstanding on said arcuate portion and disposed in a generally vertical plane and in substantially parallel relationship with each other;
(d) a contoured inner surface carried on said arcuate portion, said contoured inner surface having a generally convex shape in each of said generally vertical plane and in a direction parallel to said predetermined length, wherein at least a portion of said arcuate portion carrying said contoured inner surface thereon is manufactured from a combination of cork and latex materials enhanced with a layer of memory foam;
(e) a pouch having each of a hollow interior and an opening formed through a wall or edge of said pouch for inserting into said hollow interior or removing therefrom at least one of a cold pack, a heat pack and a gel pack;
(f) a fastener for selectively opening and closing said opening in said pouch; and
(g) a hinge securing one end of said pouch to a free end of one of said pair of legs.

17. The apparatus, according to claim 16, wherein said apparatus includes a second pouch having each of a hollow interior and an opening formed through a wall or edge of said second pouch for inserting into said hollow interior of said second pouch or removing therefrom said at least one of said cold pack and heat pack and wherein one end of said second pouch is secured with said hinge to said one of said pair of legs.

18. An apparatus for applying at least one of cryotherapy and thermotherapy to at least a portion of a knee region of an elevated leg, said apparatus comprising:
(a) a first member;
(b) a second member having a predetermined length and a generally U-shaped cross-section in a direction transverse to said predetermined length, said U-shaped cross-section defined by each of an arcuate portion mounted on a top end of said first member and a pair of spaced apart legs thereof disposed in a generally vertical plane and upstanding on said arcuate portion;
(c) a contoured inner surface carried on said arcuate portion, said contoured inner surface having a generally convex shape in each of said generally vertical plane and in a direction parallel to said predetermined length; and
(d) means engageable with each of said second member and the at least portion of the knee region for applying at least one of cold and heat thereto.

19. The apparatus, according to claim 18, wherein at least a portion of said arcuate portion carrying said contoured inner surface thereon is manufactured from a combination of cork and latex materials enhanced with a layer of memory foam.
